(19) United States
(12) Patent Application Publication
(10) Pub. No.: US 2006/0104934 A1
(43) Pub. Date: May 18, 2006

(54) Cosmetic composition comprising ubiquitin

(76) Inventors: Christian Lubrano, Issy Les Moulineaux (FR); Jean-Renaud Robin, Nanterre (FR)

Correspondence Address: YOUNG & THOMPSON, 745 SOUTH 23RD STREET, 2ND FLOOR, ARLINGTON, VA, 22202, US

(21) Appl. No.: 10/521,197

(22) PCT Filed: Jul. 7, 2003
(86) PCT No.: PCT/FR03/02109

§ 371 Date: Sep. 22, 2005

(30) Foreign Application Priority Data
Jul. 16, 2002 (FR) 02/08992

(51) Int. Cl. A61K 8/64

(52) U.S. Cl. 424/70.14

(57) ABSTRACT

The invention relates to a cosmetic composition comprising ubiquitin in combination with a cosmetically-acceptable medium.

The present invention relates to a cosmetic composition comprising ubiquitin, that is useful in particular for protecting the skin against outside attacks and combating skin aging.

Ubiquitin (Ub) is a small protein of 76 amino acids. This protein is found in all eukaryotic organisms, and is highly conserved from one species to another. Thus, there are only three differences in the yeast ubiquitin sequence compared with the human sequence.

It is a ubiquitous protein and is involved in several cell processes.

Ubiquitin is in particular involved in the regulation of proteins. The conjugation thereof to abnormal proteins or proteins to be eliminated constitutes a signal for destruction by certain cell systems, such as the proteasome 26 system, which then ensure degradation of these proteins.

Ubiquitin is also considered to be an HSP (heat shock protein), insofar as it is synthesized in the event of cell stress.

The authors of the present invention now propose to use this protein in the cosmetics field, for increasing the threshold of tolerance of the skin with respect to stresses induced by outside attacks (U.V., pollution) and for combating skin aging.

One of the desired effects is in particular the protection of the keratinocytes, which are cells of the epidermis, and the fibroblasts, which are cells of the dermis, which produce the collagen and elastin, which are fibers that support the dermis. Protecting these cells therefore amounts to ensuring a relatively continuous synthesis of these fibers that are essential to the skin's resistance.

A subject of the present invention is therefore a cosmetic composition comprising ubiquitin in combination with a cosmetically acceptable vehicle.

The cosmetic composition of the invention is intended to be applied to the skin.

Advantageously, the ubiquitin is present in a proportion of 0.001 to 0.1% by weight of the composition, preferably of 0.005 to 0.5%, preferably 0.01%.

The ubiquitin used may be of any origin, may be synthetic or may be extracted from an organism. Preferably, the ubiquitin is of plant origin, and may, for example, be obtained from plants rich in proteins, such as alfalfa, but also from cereal sources such as rice, wheat or dry vegetables such as beans or peas.

The ubiquitin may in particular be provided in the form of a plant extract, preferably at least partially purified, containing at least 1%, preferably from 1 to 10%, by weight relative to the solids.

The ubiquitin may be extracted from whole plants, from specific plant organs (for example seeds, leaves, etc.) and/or from in vitro plant cell cultures.

The extraction may be carried out by means of conventional solvents for extracting proteins, in particular saline (in particular NaCl) solutions. The purification methods may be envisioned, for example, among the following techniques: fractionated precipitation with salts (ammonium sulfate, for example) of varying concentration or with solvents such as ethanol optionally diluted in water, separation techniques, preparative ion exchange chromatography. These techniques may be combined in order to obtain the desired degree of purification.

According to a particular embodiment, a ubiquitin concentrate is obtained by:
grinding of the plant (for example alfalfa), and extraction with stirring in a sodium citrate buffer, at pH 5. A protease inhibitor is advantageously added in order to prevent degradation of the ubiquitin;
filtration of the extract and heating to 85° C. for the purpose of precipitating the thermolabile proteins. The heat-resistant ubiquitin remains in solution;
separation of the supernatant by centrifugation or tangential filtration through membranes;
further precipitation by adding ammonium sulfate;
separation of the precipitate (by centrifugation);
washing of the recovered precipitate.

An example of ubiquitin purification is described in Vierstra et al., Journal of Biological Chemistry, 1985, 260(22):12015-12021.

The cosmetic compositions according to the invention are preferably provided in the form of a simple oil-in-water or water-in-oil emulsion, or of a multiple emulsion, of a microemulsion, of an aqueous or aqueous-alcoholic gel, of a cream, of an oil, or of an aqueous or aqueous-alcoholic lotion.

The invention also relates to a cosmetic treatment method, comprising the application, to an area of the skin to be treated, of a cosmetic composition as defined above.

These cosmetic compositions are particularly advantageous for combating skin aging, i.e. in particular the phenomena of wrinkles, and of loss of tonicity and of elasticity due to structural modifications of the skin because of age.

The cosmetic compositions of the invention are also useful for protecting the skin against external attacks, such as in particular ultraviolet rays or air pollution.

The following examples illustrate the invention without limiting the scope thereof.

### EXAMPLE 1

### Formulation of an Anti-Wrinkle Cream

| Ubiquitin | 0.01% or dry extract |
|---|---|
| | containing 1% of |
| | ubiquitin: 1% |
| Demineralized water | QS |
| Urea | 4% |
| Na4 EDTA | 0.025% |
| Gluceth-20 stearate | 2.5% |
| Xanthan gum | 0.8% |
| Propylene glycol | 1.5% |
| Polyglycol 400 | 5.5% |
| Grapeseed oil | 4.5 |
| Sweet almond oil | 2% |
| Cameline oil | 1.5% |
| Preserving agents | 1.5% |
| Glyceryl stearate | 0.5% |
| Stearyl alcohol | 3% |
| Soybean lecithin | 0.1% |
| Tocopheryl acetate | 0.1% |
| 5 cyclomethicone | 3% |
| Water-glycol extract of horsetail | 5% |
| Water-glycol extract of walnut | 4% |
| Fragrance | 0.3% |

### EXAMPLE 2

### Formulation of a Nighttime Care Cream

| Ubiquitin | 0.01% or dry extract |
|---|---|
| | containing 1% of |
| | ubiquitin: 1% |
| Demineralized water | QS |
| Na4 EDTA | 0.025% |
| Propylene glycol | 3% |
| 1% Silanol lactate | 1.7 |
| Silanol mannuronate | 2.3 |
| D-panthenol | 0.2% |
| Xanthan gum | 0.1% |
| Glyceryl stearate | 2.8% |
| Steareth 20 | 5.5% |
| Cetyl alcohol | 3.0% |
| Ethylhexyl cocoate | 4.0% |
| Tocopheryl acetate | 0.05% |
| Dimethicone | 1.5% |
| Jojoba oil | 3.0% |
| Sunflower oil | 3.0% |
| Karite butter | 4.5% |
| Preserving agents | 1.5% |
| Urea | 3.5% |
| Fragrance | 0.2% |

### EXAMPLE 3

### Formulation of a Lotion

| Ubiquitin | 0.01% or dry extract |
|---|---|
| | containing 1% of ubiquitin: 1 |
| Water | QS |
| EDTA | 0.05% |
| Propylene glycol | 2.0% |
| Preserving agents | 1.5% |
| Alcohol | 25% |
| Oleyl alcohol with 20 EO | 1.0 |
| Fragrance | 0.05% |

1-7. (canceled)

8. A cosmetic composition intended to be applied to the skin, comprising ubiquitin in combination with a cosmetically acceptable vehicle.

9. The cosmetic composition as claimed in claim 8, in which the ubiquitin is present in a proportion of 0.001% to 0.1% by weight of the composition.

10. The cosmetic composition as claimed in claim 8, in which the ubiquitin is of plant origin.

11. The cosmetic composition as claimed in claim 10, in which the ubiquitin is in the form of a plant extract containing at least 1% of ubiquitin by weight relative to the solids.

12. The cosmetic composition as claimed in claim 10, in which the ubiquitin comes from alfalfa.

13. A cosmetic treatment method, comprising the application, to an area of the skin to be treated, of a cosmetic composition as claimed in claim 8.

14. The method of claim 13, for combating skin aging and protecting the skin against external attacks.
